(19) United States
(12) Patent Application Publication
(10) Pub. No.: US 2010/0144890 A1
(43) Pub. Date: Jun. 10, 2010

(54) USE OF A SURFACTANT FOR THE PREPARATION OF A FORMULATION FOR THE TREATMENT OF ADIPOSE DISEASES

(76) Inventors: Peter Boderke, Schwalbach (DE); Bernhard Hauptmeier, Glenhausen (DE); Edgar Mentrup, Frankfurt Am Main (DE); Holger Kohler, Ingelheim (DE); Rainer Pooth, Bad Soden/Taunus (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 12/449,003

(22) PCT Filed: Jan. 14, 2008
(86) PCT No.: PCT/EP2008/050350

§ 371 Date: Dec. 15, 2009

(30) Foreign Application Priority Data
Jan. 17, 2007 (EP) 07000945.1
Mar. 14, 2007 (EP) 07005271.7

(51) Int. Cl. A61K 31/10; A61K 31/21; A61K 31/075

(52) U.S. Cl. 514/709; 514/506; 514/715

(57) ABSTRACT

Use of a mixture comprising at least one non-aromatic surfactant having a plurality of ether and/or ester groups and at least one carrier agent for the preparation of a formulation for the treatment of adipose tissue disease and/or condition with improved bioavailability and lipolysis behaviour.

The present invention relates to the use of a surfactant for the manufacturing of a product for the treatment of adipose diseases and/or conditions.

In general, aqueous, hydrophilic mixtures comprising active ingredients, surfactants and lipophilic substances are well known in the field of pharmaceutical formulations as emulsions and micro-emulsions. Emulsions act as stable carrier for drugs that show poor water solubility. In such known mixtures there is always a drug or active ingredient that dictates the choice of the other components. The mixtures or pharmaceutical compositions should be tailor made with regard to the desired mode of action, the intended route of application and should stabilise the active ingredient against physical, chemical or microbiological degradation.

Thus, any effect of the other components of the emulsion or micro-emulsion formulation other than the carrier function is undesired.

On the other hand, aqueous systems of phospholipids and bile acid or its derivatives are well known for the preparation of cosmetic and pharmaceutical formulations.

EP 0 615 746 A1 and WO 2005/112942 A1 describe such formulations that can carry a pharmaceutically active substance or that can be used without such an active drug. In the latter case, it is described that such liposomes can be used for the treatment of atherosclerosis, elevated blood lipids, and hepatopathy of any kind.

The described systems show a distinct liposome structure, i.e. a double membrane of lipids that encapsulates an aqueous phase.

In recent literature it is further described that such liposome systems of phosphoslipids and bile acid can reduce fatty tissue when locally injected subcutaneously (Patricia Guedes Rittes, The use of phosphatidylcholine for correction of lower lid bulging due to prominent fat pads, Dermatol Surg 2001, 27, 391-392).

Further, a special liposome system for the prophylaxis and treatment of fatty embolism is known that comprises components such as phosphatidylcholin, bile acid, DL-alpha-Tocopherol, ethanol and water (Lipostabil® N i.V.).

However, the known aqueous liposome systems of phospholipids and bile acid or its derivatives for the treatment of reducing fatty tissue have the distinct disadvantage that their distribution inside the tissue is poor and thus the effect is fairly locally constricted to the immediate point of injection. Apart from that, it could be seen that the known active ingredients like phosphatidylcholin or desoxycholate can show side effects like localized inflammation and others which make the use of those systems for injection treatment uncomfortable. Accordingly, up to date it is necessary for the treatment of a larger area of tissue to apply a high number of injections close to each other.

Therefore, it is an object of the present invention to provide a formulation for the treatment of adipose diseases and/or conditions that shows a good biocompatibility and an enhanced efficacy, especially bioavailability such that larger areas of fatty tissue can be treated while generally being able to show less side effects. The term bioavailability according to this patent application is used in view of the target tissue to be treated.

Surprisingly, it was found that the use of a mixture comprising at least one non-aromatic surfactant having a plurality of ether and/or ester groups and at least one carrier agent for the preparation of a formulation for the treatment of adipose tissue disease and/or condition meets the object of the present invention.

The present invention furthermore relates to the use of a mixture comprising at least one non-aromatic surfactant having a plurality of ether and/or ester groups and at least one carrier agent for the treatment of adipose diseases and/or conditions.

The present invention furthermore relates to a process comprising: administering a mixture comprising at least one non-aromatic surfactant having a plurality of ether and/or ester groups and at least one carrier agent to a human in an amount effective for treating adipose diseases and/or conditions.

The term “carrier agent” in accordance with the present invention includes all possible solvents, solvent mixtures, and other substances being able to support, enhance or enable the application and/or transport of the surfactant to the target tissue.

The inventive use of the surfactant mixture of the present invention shows a better efficacy, in most applications also a better bioavailability and therefore a better distribution in the fatty tissue than the known lipolysis systems comprising additional ingredients like phospholipids, plant extracts, nicotine derivatives, flavonoids, and bile acid derivatives being described as active ingredients.

Apart from that, it shows improved reactivity against subcutaneous localized fat cells while being able to reduce the side effects caused by those additional ingredients of the prior art that are lacking in the mixture of the present invention. Thus, the mixture of the present invention allows for fewer injections in greater distance when a larger area of tissue is to be treated and in general, a better effect of lipolysis resulting in a higher patient convenience.

According to a preferred embodiment of the present invention, the mixture consists of at least one non-aromatic surfactant having a plurality of ether and/or ester groups and at least one carrier agent.

According to a preferred embodiment of the present invention, the at least one surfactant comprises polyether groups. This has been shown to increase the efficacy of the mixture for many applications. The term “polyether” is intended to include that ether groups are present in the surfactant's molecular structure in an oligomeric and/or polymeric fashion, e.g. as polyglycols.

Suitable and insofar preferred polyether groups are polyethylen glycol, (1,2)-polypropylen glycol and/or 1,3-polypropylenglycol units as well as substituted derivatives of these units.

It should be noted that according to a preferred embodiment of the present invention polyether and at least one, preferably a plurality of ester groups are comprised in the molecular structure of the at least one non-aromatic surfactant.

According to a preferred embodiment, the at least one non-aromatic surfactant comprises at least one ester group with a long-chain carboxy moiety, preferably with a carbon chain of at least 8 carbon atoms, preferably 10 to 30 carbon atoms. Especially preferred are esters derived out of oleic acids, ricinic acid, arachnoeic acids and mixtures thereof.

According to a preferred embodiment of the present invention, the at least one surfactant comprises at least one moiety derived from carbohydates, especially reduced carbohydrates.

The term “moiety” especially means and/or includes that such a structure is comprised somewhere in the molecular structure of the surfactant.

Especially preferred are moieties which derive from reduced 5- or 6-membered carbohydrates, especially from sorbitol. In this context it is especially preferred that in the surfactant's molecular structure at least 3, preferably 4, most preferred 5 or 6 of the hydroxy groups present in the sorbitol are modified, especially ethered or estered.

It is especially preferred that such moieties are present in case no polyether groups are comprised in the surfactant's molecular structure.

According to a preferred embodiment of the present invention, the at least one surfactant can be selected from the group of polysorbates, ethers of ethoxylated alcohols and alkyl-alcohols (C6-C16), alkyl-ester with C8-C20 with ethoxylated alcohols, ester of saturated and unsaturated acids with C8-C20 with sugars, alkylethersulfates like polyether of castor oil and ethylene oxide (e.g. cremephor EL), polyoxyethylene fatty alcohol ether, polysorbic monoester, poloxamer, poloxamine or mixtures thereof

The carrier agent can for instance be chosen from the group consisting of water, alcohols, organic solvents, oils, and mixtures thereof.

Preferably, according to one embodiment of the invention the carrier agent is water. In this embodiment, the water as carrier agent can also contain isotonizing agents like NaCl, KCl, CaCl₂, MgSO₄, NaH₂PO₄, amino acids, and the like. In addition, the isotonizing agent of the mixture of the present invention preferably can also be chosen from the group consisting of mannitol, lactose, dextrose, sorbitol, xylitol, and glycerol.

It is thought that the advantageous effects especially in view of an aqueous mixture according to the present invention are derived due to the differences in the structure of the inventive surfactant system. This is related to the lack of any ingredient formerly being seen as lipolysis active, its possible distinct properties like thermo dynamical stability and distribution as well as size of different phase droplets compared to the liposome systems having micelle structures filled with the lipophilic substance of active ingredients of the state of the art. Additionally, special colligative properties of the surfactant system could be seen to be relevant for the improvements. However, the transport mechanisms involved are not well understood so that scientifically sound evidence for the mechanism is yet to be found.

Preferably, according to one embodiment of the present invention, the surfactant has a HLB value between 4 and 20 according to the Griffin HLB-system.

In the term “HLB value” HLB stands for hydrophile-lipophile balance. Surfactants with a low HLB are more lipophilic and thus tend to make a water in oil emulsion while those with a high HLB are more hydrophilic and tend to make an oil in water emulsion. The HLB value of each surfactant is determined by an analysis of the characteristics of the surfactant. A list of HLB values for various surfactants is available in many references such as the Handbook of Pharmaceutical Excipients, 3rd Edition. The HLB value can be used to predict the surfactant properties of a molecule, typically a value from 3 to 6 indicates a W/O emulsifier, a value from 7 to 9 indicates a wetting agent, a value from 8 to 12 indicates an O/W emulsifier, a value from 12 to 15 is typical of detergents, and a value of 15 to 20 indicates a solubiliser or hydrotrope.

Preferably, the surfactant of the present invention has a HLB value of 5 to 20.

More preferably, the surfactant has an HLB value of 7 and 18, even more preferably between 9 and 17.

Examples of preferred surfactants used in mixtures according to the present invention include polysorbate 20, polysorbate 40, polysorbate 60, polysorbate 80, sorbitan monooleate, and polyether of castor oil and ethylene oxide.

In another preferred embodiment of the present invention no lipophilic and/or other lipolysis-active substance than the at least one surfactant and optionally the isotonizing agent is present in the aqueous mixture used for the preparation of a formulation for the treatment of adipose tissue disease and/or condition.

Due to the lack of such substances like plant extracts, nicotine derivatives, flavonoids, phospholipids and bile acid derivatives in the mixture and thus in the micelle and/or (micro)-emulsion structure especially of the aqueous surfactant mixture an even better uptake of the surfactant in the cells to be treated might take place. Possibly, size effects and/or colligative effects in the mixture of the present invention could be relevant for the more effective lipolysis behaviour observed in the in vitro examples. Thus, larger areas of tissue can be affected by a single injection.

In a preferred embodiment of the present invention the system further comprises at least one co-surfactant.

According to a preferred embodiment, the co-surfactant is less lipophilic than the surfactant with an HLB value of 9 to 17.

Like that, a further increase in the lipolysis of adipocytes of the inventive mixture can be achieved resulting in a shorter and more effective treatment.

The co-surfactant can in particular be chosen from the group of polysorbates, ethers of ethoxylated alcohols and alkyl-alcohols (C6-C16), alkyl-ester with C8-C20 with ethoxylated alcohols, ester of saturated and unsaturated acids with C8-C20 with sugars, alkylethersulfates like polyether of castor oil and ethylene oxide (cremephor EL), polyoxyethylene fatty alcohol ether, polysorbic monoester, poloxamer, poloxamine.

According to another preferred embodiment of the present invention the mixture additionally comprises (as part of the carrier agent and/or (co)-carrier agent) one cosolvent or a combination of cosolvents. Cosolvents in accordance with the present invention are defined as water miscible components or excipients. Examples for cosolvents are alcohols, ethers and esters of organic substances like ethanol, acetone, isopropanol, glycerine, propylene glycol, dimethylsulfoxide, dimethylacetamide, dimethylacetamide, N-methylpyrrolidone, dimethylisosorbid, 2-Pyrrol, 2-Pyrrolidone and diethyleneglycolmonoethylether.

According to another preferred embodiment of the present invention the mixture additionally comprises an alcohol.

Particularly preferred alcohols are C2-C8 alcohols, and in particular ethanol, propylene glycol, isopropanol, and glycerine.

Other organic solvents like acetone are also possible (co)-carrier agents in accordance with the present invention.

The mixture used in accordance with the present invention can additionally comprise at least one buffer.

Examples of buffers suitable to be used in a mixture of the present invention include phosphate buffer, trometamole buffer, acetate buffer, citrate buffer, tartrate buffer, carbonate buffer, lactate buffer, and glycine buffer.

In another preferred embodiment of the present invention the mixture can additionally comprise an anti-oxidant. Suitable examples of anti-oxidants that can be used in the mixture of the present invention include ascorbic acid and its derivatives, sodium sulfite, monothioglycerol, cystein, sodium dithionite, and tocopherole and its derivatives, hydroxyl anisole derivatives, alkyl gallates, thioglycolic acid.

In accordance with another preferred embodiment of the present invention the mixture can additionally comprise a local anaesthetic agent. For example, lidocaine, procaine, tetracaine, etidocaine, mepivacaine, bupivacaine, prilocaine, and/or butanilicaine can be included as local anaesthetic agent in the mixture of the present invention.

Additionally or alternatively, at least one complex builder, anti-foaming agent, and/or antimicrobial agent can be comprised in a preferred embodiment of a mixture of the present invention.

Examples for such additive substances like complex builders, anti-foaming agents, and/or conservation agents are EDTA, Dimethicone, phenol, cresol and its derivatives, benzoic acid, PHB ester, and/or sorbic acid.

In a preferred embodiment of the present invention the concentration of the surfactant in the aqueous mixture is preferably between 0.01 and 60 weight %, preferably between 0.5 and 50 weight %, more preferably between 0.8 and 30 weight %, and in particular between 1 and 25 weight % of the mixture.

The pH value of the system according to the present invention is neutral and ranges preferably between 4.0 and 9.0, more preferably between 6.0 and 8.0. The pH value can be adjusted by the possible use of acids and bases like hydrochloric acid, phosphoric acid, sulphuric acid, sodium hydroxide, calcium hydroxide and/or potassium hydroxide.

Under the term adipose tissue disease and/or condition in particular any unwanted local fat deposits and/or the following disease examples including unaesthetic appearances like cellulite are understood:

Lipomae are benign slow growing tumors of fat cells, preferred located in the subcutaneous fatty tissue that can occur in various forms and characteristics. They can build mucus, chalk and/or become ossified. Additionally, increased built of connective tissue and capsules can occur together with newly built blood vessels which are all classified as abnormal because the compression on the blood vessels as well as on the nerve cells is algetic. Lipomae occur in various syndromes like for example the Gardner syndrome, the Lanois-Bensaude syndrome, and the Proteus syndrome.

Lipomatosis dolorosa and cellulite are special forms of hypertrophic proliferation of fatty tissue which is located between the dermal fatty fascia and the underside of the dermis. Due to hormonal influences an enhanced capability to bind water in these fatty cells is observed which themselves initiate pressure and cause subsequently congestions in the lymphatic vessels. Additionally, compression and irritation to the peripheral sensitive nerves is applied so that the patients have an extreme sensitivity to contact. Over the years, irregular disseminated localised fatty nodes can built under the thinning dermis which are painful and show an unaesthetic character.

In this context also conditions like Lipoedema or lipodystrophic syndrome have to be mentioned.

The above addressed fatty tissue diseases demonstrate in contrast to alimentary related adipose disease pathophysiological tissue conditions that can be identified by histological scar and inflammation parameter as well as modifications in the histological fatty tissue morphology.

Under the term regression it is in particular understood that the lipolysis of the fatty tissue and the degeneration of the prolific fatty tissue is taking place.

In another preferred embodiment of the present invention the aqueous mixture of at least one surfactant, at least one isotonizing agent and water is used in form of a micro-emulsion.

Micro-emulsions generally are clear, isotropic liquid mixtures of water, oil and surfactant.

The water phase may contain salts and/or other ingredients. It is possible to prepare micro-emulsions from a large amount of components. In contrast to ordinary emulsions micro-emulsions form upon simple mixing of the components and do not require high shear conditions. In ternary systems such as micro-emulsions where two immiscible phases (water and “oil”) are present next to the surfactant phase, the surfactant molecules form a monolayer at the interface between oil and water. The hydrophobic part of the surfactant molecules are dissolved in the oil phase and the hydrophilic part of the surfactant molecules are in the aqueous phase. Micro-emulsions are thermodynamically stabilized by the surfactant in a special way because they are not simply a dispersion of droplets of oil in water or vice-versa but a more complex mixture of solute, solution, reversed and normal micelles, and micro-emulsion droplets.

The droplet size of the micro-emulsions of the above embodiment of the present invention is preferably between 10 nm and 200 nm, more preferably between 30 nm and 100 nm.

The micro-emulsion according to the present invention is preferably transparent or light opaque. Micro-emulsions are single phased in a given range of pressure, temperature, and composition. In contrast to emulsions they are thermodynamically stable systems due to their small particle sizes and they have the advantage that they build spontaneously and are stable even if stored for a long time.

Subject to the type of surfactant used micro-emulsions are distinguished into ionic and non-ionic micro-emulsions.

The treatment with a formulation of the present invention is preferably directed to cellulite tissue and/or local deposits of unaestethic fatty tissue. In contrast to the known treatments especially the areas of mainly unaesthetic character are very receptive to the beneficial effect of a acceptable biocompatibility and an enhanced efficacy such that wider areas can be reached with a single application.

In general, all unwanted and/or unaesthetic fatty tissue can be treated with the formulations of the present invention. This includes adipose tissue around the eyes, at the cheeks, in the neck and chin region, at the back, under and around the arms, at the thighs, in the upper and lower stomach region, at the knee, and/or so called lovehandles by males, gluteal bananas by females, and saddlebacks.

In particular, with the use of aqueous surfactant mixtures according to the present invention local deposits of unaesthetic fatty tissue around the eyes, under the arms, in the neck and chin region and/or at the tights are preferably treated. Those body regions often show a high sensitivity so that a possible reduction of injection points and injection frequency is most beneficial especially for the tissue in the mentioned regions.

The preparation of a mixture of the present invention can for instance be such that at least one surfactant and at least one isotonizing agent are mixed in water in a ratio disclosed above. The preparation can be brought forward by any known form of preparation of aqueous mixtures.

Application of a mixture of the present invention can be carried out by any form of parenteral or topical application, in particular by subcutaneous injection.

A mixture of the present invention consisting of at least one surfactant having a HLB value between 4 and 23 and at least one carrier agent is preferably used for the preparation of an injection product for the subcutaneous treatment of adipose tissue disease and/or condition.

The carrier is preferably as described above.

According to a further aspect of the present invention it was found that the use of a mixture comprising at least one alkylsulfate and at least one carrier agent for the preparation of a formulation for the treatment of adipose tissue disease and/or condition meets the object of the present invention.

The present invention furthermore relates to the use of a mixture comprising at least one alkylsulfate and at least one carrier agent for the treatment of adipose diseases and/or conditions.

The present invention furthermore relates to a process comprising: administering a mixture comprising at least one alkylsulfate and at least one carrier agent to a human in an amount effective for treating adipose diseases and/or conditions.

The term alkylsulfate especially refers to the molecular structure R—OSO₃⁺M_{/x}^{x+}, whereby R is any alkylic residue and M is a metal ion. Preferred metal ions include Na⁺, K⁺, Mg²⁺, and Ca²⁺.

Preferably R is a long-chain alkyl residue having at least 5 carbon atoms, more preferred between 8 and 15 carbon atoms, whereby the alkyl residue may be mono, di, or polysubstituted by halogen, hydroxy and/or alkyloxy groups. Furthermore the alkyl chain may be unsaturated, i.e. according to a preferred embodiment of the present invention, the alkyl chain may comprise one or more alkene and/or alkine groups.

Especially preferred alkylsulfates comprise octylsulfate, decylsulfate and laurylsulfate.

### EXAMPLES

The effects on cytotoxicity in adipocytes using LDH release and Calcein-AM release assays of 4 liquid compounds were tested with reference products:

1 TWEEN 80, (being polysorbate 80);

2 CREMOPHOR EL (being a polyethoxylated castor oil);

3 SPAN 80 (being Sorbitan monooleate),

as reference example for a prior art system 4 LIPOSTABIL TM (being an aqueous phophatidylcholin and bile acid containing lipolysis injection solution).

Tween 80 has the following structure:

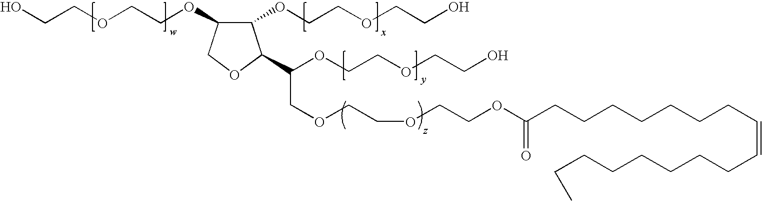

Cremophor EL has the following structure:

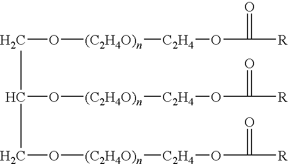

(n=11 or 12, R=ricinoleic acid)

Span 80 has the following structure:

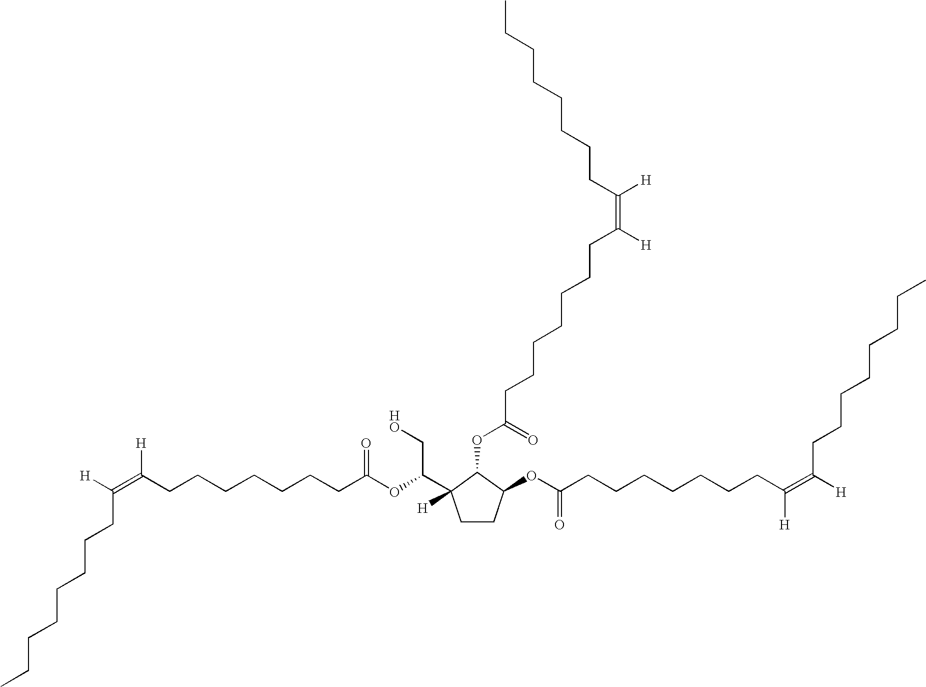

Experiments were performed in 96 wells/plates and a dilution of 1:512 suggest that 0, 37 μl of liquid compounds media were added directly to the cells in 200 μl of media.

Experimental Procedures

Cell Cultures:

Primary human preadipocytes were obtained from Promocell (Heidelberg, Germany). The Order No is C-12731 and the Lot-No 5112201 (abdomen, 44 year old Caucasian female).

Culture Protocol:

Human preadipocytes cells were maintained in Preadipocyte Growth Medium (DMEM; BioWhitaker Cat: BE12-604F/U1; Lot N° 6MB0130 containing 10% heat-inactivated FCS and 1% penicillin-streptomycin) at 37° C. in a humidified atmosphere of 5% CO2. The cells were expanded in several T75 flasks to reach 60-80% confluence. To differentiate the preadipocytes (104 cells/well) were grown in 96 well plates to reach confluence. At this point (day 0), cells were switched to differentiation medium (DMEM, 10% FBS, 0.25 μM dexamethasone, 0.25 mM IBMX, and 1 pg/mL insulin) for 3 days, with one medium change in between. On day 3, the dexamethasone and IBMX was removed leaving insulin on the cells for an additional 4 days, changing the medium every 2 days. Thereafter, the cells were maintained in the original propagation DMEM, changing medium every 2-3 days until use. Plates where cells are >75% differentiated were used for experiments (day 10 post-induction). Adipocytes are round and full of easily distinguishable fat globules after 7 days of culture.

Cytotoxicity assays: The CytoTox-ONE™ Homogeneous Membrane Integrity Assay from Promega was used to study LDH release (The Lot-No 234242/27/09/08). Differentiated adipocytes were treated with the test compounds for either 24 or 48 hours and the release of LDH detected using the fluorimetric assay CytoTox-ONE (see above) according to the manufacturer's instructions. Maximum LDH release was determined by complete lysis of cells using 0.1% Triton X-100. Control cells treated with water alone were used to determine the baseline level of LDH release (0% cell death). The fluorescence was detected in a Genius Pro apparatus using specific 96 microwells plates for fluorescence. We used excitation filters in the 530-570 nm range and emission filters in the 580-620 nm range.

Calculation of Results

The percentages cytotoxicity by LDH release were calculated according to the manufacturer's instruction (http://www.promega.com/tbs/tb306/tb306.pdf)

$$Percent cytotoxicity = 100 \times \frac{( \begin{matrix}Experimental - \\ Culture Medium Background\end{matrix} )}{( \begin{matrix}Maximum LDH Release - \\ Culture Medium Background\end{matrix} )}$$

According to this calculation the spontaneous release in untreated cells may indicate some cytotoxicity in untreated cells. This could be explained because upon confluence of pre-adipocytes some of the cells do not differentiated to adipocytes and some cytotoxicity by cell overgrowth should be expected. We usually found that in untreated confluent pre-adipocytes cells the LDH release could reach values up to 15-20% of the total LDH release induced by Triton X-100 (100% release) and in differentiated adipocytes the spontaneous LDH release can reach values up to 35% (depending of the time of culture). This type of calculation may induce to misinterpretations and therefore the specific cytotoxicity was calculated according to the following formula:

$$\% S . C . \text{:} 100 \times \frac{( \begin{matrix}\% Spontaneous LDH release - \\ \% Spontaneous LDH release\end{matrix} )}{( \begin{matrix}\% Triton \text{-} induced LDH release - \\ \% Spontaneous LDH release\end{matrix} )}$$

S.C. values below 0% were considered arbitrary as 0% of substance-induced cytotoxicity.

Cytotoxicity Assay in Adherent Cells (3T3-L1) using the Calcein-AM Method:

Cells were seeded into 96-well plates at 104 viable cells per well and left to attach to the plate for 24 h. After 24 h, the cells were stimulated as indicated for another 24 h. Under these culture conditions 3T3-L1 cells do not differentiated to adipocytes. Then, the wells were washed and the cells incubated with Calcein-AM (1 μM) (Molecular Probes) for 30 min. Then the fluorescence of viable cells was detected in a microtitre plate reader (TECAN Genius Pro). Using this method the uptake of Calcein-AM by viable cells is measured. Calcein-AM his hydrolysed by cellular esterases and then the fluorescence emitted by calcein is detected in the plate reader (excitation: 490 nm, emission: 515 nm)

Auto-fluorescence analysis of the test substances: No-cell controls with medium and test substance alone were prepared to determine the effect of the test compound on background fluorescence. We used excitation filters in the 530-570 nm range and emission filters in the 580-620 nm range. We found that none of the test substances included in this study showed auto-fluorescence.

Effects of the Test Substances on Both Preadipocytes and Differentiated Adipocytes (LDH Release):

To confirm the cytotoxic activity of the test substance at certain concentrations, undifferentiated (24 h treatment) and differentiated human primary adipocytes the cultures were treated with the indicated dilutions during 24 h and 48 h and the release of LDH measured in the supernatants.

TABLE 1
Effects of the test substances on both preadipocytes
and differentiated adipocytes (LDH release)

| | | Preadipocytes | | Adipocytes |
|---|---|---|---|---|
| # Test | | (LDH) | Differentiated | (LDH) |
| Substance | Dilution | 24 h | 24 h | 48 h |
| 1 | 1:512 | Toxic (38.1%) | Toxic (42.8%) | Toxic (100%) |
| 2 | 1:512 | Toxic (32.6%) | Toxic (56.1%) | Toxic (100%) |
| | 1:10000 | Non-toxic | Non-toxic | Slightly- |
| | | | | toxic (20.3%) |
| 3 | 1:512 | Non-toxic | Non-toxic | Non-toxic |
| | 1:10000 | Non-toxic | Non-toxic | Non-toxic |
| 4 (prior art) | 1:512 | Non-toxic | Non-toxic | Non-toxic |
| | 1:10000 | Non-toxic | Non-toxic | Non-toxic |

Cytotoxic Effects of Test Substance on Adipoytes:

Calcein-AM uptake: The cells were incubated with different concentrations of the test compounds for 24 h and the cell viability was analysed by the fluorescent Calcein-AM method. In this type of assay the higher RFU (relative fluorescent unit) correspond to viable cells.

TABLE 2
Dilutions where cytotoxicity on adipocytes is still present

| # Test | | Adipocytes (Calcein-AM) | Adipocytes (LDH) |
|---|---|---|---|
| substance | Dilution | 24 h | 24 h |
| 1 | 1:50 | Toxic | Toxic |
| | 1:100 | Toxic | Toxic |
| | 1:200 | Toxic | Toxic |
| | 1:400 | Toxic | Toxic |
| | 1:800 | Toxic | Toxic |
| | 1:1600 | Toxic (49.3%) | Non-toxic |
| | 1:3200 | Non-toxic | Non-toxic |
| | 1:6400 | Non-toxic | Non-toxic |
| | 1:12800 | Non-toxic | Non-toxic |
| 2 | 1:50 | Toxic | Toxic |
| | 1:100 | Toxic | Toxic |
| | 1:200 | Toxic | Toxic |
| | 1:400 | Toxic | Toxic (43.6%) |
| | 1:800 | Toxic (48.4%) | Non-toxic |
| | 1:1600 | Slightly-toxic (26.2%) | Non-toxic |
| | 1:3200 | Slightly-toxic (22.2%) | Non-toxic |
| | 1:6400 | Non-toxic | Non-toxic |
| | 1:12800 | Non-toxic | Non-toxic |
| 3 | 1:12.5 | Toxic | Toxic |
| | 1:25 | Toxic | Toxic |
| | 1:50 | Toxic | Toxic |
| | 1:100 | Toxic | Toxic |
| | 1:200 | Toxic | Toxic (34.9%) |
| | 1:400 | Toxic | Non-toxic |
| | 1:800 | Slightly-toxic (34.9%) | Non-toxic |
| | 1:1600 | Non-toxic | Non-toxic |
| | 1:3200 | Non-toxic | Non-toxic |
| Reference | 1:12.5 | Toxic | Toxic |
| 4 | 1:25 | Toxic | Toxic |
| | 1:50 | Toxic | Toxic |
| | 1:100 | Toxic | Toxic |
| | 1:200 | Toxic | Toxic (25.9%) |
| | 1:400 | Toxic (50%) | Non-toxic |
| | 1:800 | Non-toxic | Non-toxic |
| | 1:1600 | Non-toxic | Non-toxic |
| | 1:3200 | Non-toxic | Non-toxic |

As can be seen from table 2 the effect of the use of aqueous mixtures according to the present invention in lipolysis of adipocytes is derived even at a much higher dilution than the reference example being a phosphatidylcholin system (Lpostabil TM).

On the basis of the above examples dilutions were calculated at which 50% were still alive in case of the Calcein assay or which generated 50% cell death in case of the LDH assay. The results are shown in the following table 3:

| | Calcein assay | |
|---|---|---|
| | Dilution at which | LDH assay |
| # Test | 50% of the cells are | Dilution generating 50% |
| Substance | alive | cell death |
| 1 | 1:1631 | 1:328 |
| 2 | 1:793 | 1:184 |
| 3 | 1:724 | ND* |
| 4 (prior art) | 1:438 | 1:32 |

*ND: No curve could be established for the LDH assay. Around 30% of the cells were dead with concentrations ranging from 1:12.5 to 1:200.

Furthermore effects on cytotoxicity in adipocytes using LDH release and Calcein-AM release assays of sodium laurylsulfate was tested with the same test procedure as described above.

| | Adipocytes (Calcein-AM) | Adipocytes (Calcein-AM) |
|---|---|---|
| Dilution | 6 h | 24 h |
| 1:4000 | Toxic | Toxic |
| 1:16,000 | Toxic | Toxic |
| 1:512000 | Non-Toxic | Non-Toxic |

Therefore sodium laurylsulfate is even more potent that the other inventive substances of the present invention.

1-22. (canceled)

23. A method for the treatment of an adipose tissue disease and/or condition comprising administering to a human or animal subject, an effective amount of a preparation comprising at least one non-aromatic surfactant having a plurality of ether and/or ester groups and at least one carrier agent.

24. The method of claim 23, wherein the at least one non-aromatic surfactant has a hydrophile-lipophile balance (HLB) value between 4 and 20.

25. The method of claim 23, wherein the at least one non-aromatic surfactant comprises polyether groups.

26. The method of claim 23, wherein the at least one non-aromatic surfactant comprises at least one ester group with a carboxy-group having a long-carbon chain.

27. The method of claim 23, wherein the at least one non-aromatic surfactant comprises at least one moiety derived from a carbohydrate.

28. The method of claim 23, wherein the at least one non-aromatic surfactant comprises a moiety derived from sorbitol.

29. The method of claim 23, wherein the at least one non-aromatic surfactant is selected from polysorbates; ethers of ethoxylated alcohols and alkyl-alcohols (C6-C16); alkyl-ester with C8-C20 with ethoxylated alcohols; ester of saturated and unsaturated acids with C8-C20 with sugars; alkylethersulfates including polyether of caster oil and ethylene oxide (cremephor EL); polyoxyethylene fatty alcohol ether; polysorbic monoester; poloxamer; and poloxamine.

30. The method of claim 23, wherein the preparation comprises at least one alkylsulfate and at least one carrier agent.

31. The method of claim 30, wherein the alkylsulfate is selected from octylsulfate, decylsulfate and laurylsulfate.

32. The method of claim 23, wherein the carrier agent comprises at least one isotonizing agent selected from sodium chloride, mannitol, lactose, dextrose, sorbitol, xylitol, and glycerol.

33. The method of claim 23, wherein the carrier agent is a mixture of water and an isotonizing agent.

34. The method of claim 23, wherein the preparation additionally comprises at least one co-surfactant.

35. The method of claim 23, wherein the preparation additionally comprises at least one buffer.

36. The method of claim 23, wherein the preparation additionally comprises at least one anti-oxidant agent.

37. The method of claim 23, wherein the preparation additionally comprises at least one local anaesthetic agent.

38. The method of claim 23, wherein the preparation additionally comprises at least one complex builder, anti-foaming agent, and/or conservation agent.

39. The method of claim 23, wherein no lipophilic and/or other lipolysis-active substance is present in the preparation.

40. The method of claim 23, wherein the treatment of an adipose tissue disease and/or condition is directed to cellulite tissue and/or local deposits of unaestethic fatty tissue.

41. The method of claim 40, wherein the local deposits of unaesthetic fatty tissue are around the eyes, under the arms, in the neck and chin region, and/or at the thighs.

42. A method for the treatment of an adipose tissue disease and/or condition comprising subcutaneously administering to a human or animal subject, an effective amount of an injection product comprising of at least one non-aromatic surfactant having a plurality of ether and/or ester groups and at least one carrier agent for the subcutaneous treatment of adipose tissue disease and/or condition.

43. A preparation comprising at least one non-aromatic surfactant having a plurality of ether and/or ester groups and at least one carrier agent which is administered to a human or animal subject in an amount effective for the treatment of an adipose tissue disease and/or condition.
